(19) United States
(12) Patent Application Publication
(10) Pub. No.: US 2025/0288805 A1
(43) Pub. Date: Sep. 18, 2025

(54) RETURN ELECTRODE SELECTION TO AVOID OFF-TARGET EFFECTS

(71) Applicant: The Alfred E. Mann Foundation for Scientific Research, Valencia, CA (US)

(72) Inventor: Brian SHELTON, Ventura, CA (US)

(21) Appl. No.: 19/081,948

(22) Filed: Mar. 17, 2025

Related U.S. Application Data
(60) Provisional application No. 63/566,010, filed on Mar. 15, 2024.

(51) Int. Cl. A61N 1/36; A61N 1/05

(52) U.S. Cl. CPC A61N 1/36053; A61N 1/0556; A61N 1/36139; A61N 1/36146

(57) ABSTRACT

The present disclosure provides systems and methods for providing vagus nerve stimulation, including configurations wherein stimulation is provided by a VNS stimulator used to transmit electrical stimulation pulses to a vagus nerve of the subject using an electrode stimulation cuff comprising a circumferential array of electrodes. In some aspects, stimulation is provided by selecting an electrode in the circumferential array of electrodes as a cathode electrode; causing the stimulator to transmit an electrical stimulation pulse to the vagus nerve using the selected cathode electrode, while (i) at least one other electrode in the circumferential array of electrodes is used as a return electrode, or (ii) all other electrodes in the circumferential array of electrodes are used as return electrodes; and repeating this process at least once, wherein a different electrode in the circumferential array of electrodes is selected as the cathode electrode.

### CROSS-REFERENCE TO RELATED APPLICATION

This application claims the benefit of U.S. Provisional Application No. 63/566,010, entitled “RETURN ELECTRODE SELECTION TO AVOID OFF-TARGET EFFECTS,” which was filed on Mar. 15, 2024, the entire contents of which is expressly incorporated by reference herein in its entirety.

### BACKGROUND

Epilepsy and depression are two extremely common maladies. Epilepsy produces potentially-fatal seizures. Both conditions can be treated under appropriate circumstances with vagus nerve stimulation (“VNS”). VNS entails the surgical implantation of a stimulator device into a patient's chest area under the skin to stimulate the vagus nerve with electrical stimulus pulses. The vagus nerve originates from the brainstem and traverses both sides of the neck down to the chest and abdomen. The VNS device sends electrical signals via the vagus nerve to the brain. A lead wire having a cuff at the proximal end connects the stimulator device to the vagus nerve. The cuff has one or more electrodes within the cuff and, when implanted, encircles the vagus nerve. VNS has been shown to be helpful in many cases for reducing the number and severity of seizures, particularly for patients who are less responsive to more non-invasive methods like oral medication. VNS has also been shown to reduce depression in certain treatment-resistant patients.

Peripheral nerve fibers, such as those of the vagus nerve, are grouped based on the diameter, signal conduction velocity, and myelination state of the axons. Fibers of the type-A group have a large diameter, high conduction velocity, and are myelinated. Type-A fibers are further organized into Aα, Aβ, Aδ, and Aγ subtypes, which are each associated with different functionalities (e.g., control of motor neurons, and the sensation of touch, pressure, pain). Fibers of the type-B group (e.g., preganglionic autonomic nerves) are myelinated with a small diameter and have a low conduction velocity. The primary role of B fibers is to transmit autonomic information. Fibers of the C group are unmyelinated, have a small diameter, and low conduction velocity.

While VNS is known to provide therapeutic and other benefits, present stimulation methods suffer from several drawbacks, including the inability to achieve selective stimulation of particular nerve types. For example, stimulating a nerve bundle such as a vagus nerve will normally recruit type-A nerve fibers before type-B nerve fibers are recruited. As a result, prior systems and methods are unable to selectively stimulate preganglionic sympathetic type-B fibers without creating an action potential on all type-A motor neurons in the same nerve.

### BRIEF SUMMARY OF EXEMPLARY ASPECTS OF THE DISCLOSURE

The present disclosure provides stimulation systems, and related methods of treatment, that utilize a multi-electrode nerve cuff that may be used to selectively stimulate type-B nerve fibers while avoiding or minimizing stimulation of type-A nerve fibers in the same nerve. Such systems and methods may be used, e.g., to provide VNS while minimizing undesirable side effects that result from the activation of type-A motor neurons e.g. laryngeal electromyography (“EMG”) activation or cardiac response such as tachycardia or bradycardia. As explained herein, the present systems utilize one or more nerve cuffs that each comprise a circumferential array of electrodes which encircle (or substantially encircle) a nerve such as the vagus nerve. A controller of the stimulation system may be used to trigger stimulation of a first subset of the circumferential array of electrodes (as cathode electrodes(s)) while a second subset of the circumferential array of electrodes are used as a return (as anode electrode(s)). In some embodiments, stimulation of the circumferential array of electrodes may be delivered as periodic stimulation. In some embodiments, the return (anode) electrode is chosen to be the electrically conductive metal housing portion of an implantable VNS stimulator. In doing so, stimulation may be focused on particular nerve fibers located within the cuffed nerve. In some aspects, the amplitude of stimulation (or other stimulation parameters) may be independently set for each cathode electrode or each set of cathode electrodes in order to further customize treatment for a subject. Moreover, as described herein and illustrated by the provided figures, the present systems may be configured to provide periodic stimulation according to multiple patterns For example, stimulation may be applied by sequentially alternating between a set of cathode electrodes, or by simultaneously stimulating all of a set of cathode electrodes, while one or more anode electrodes are maintained as a return electrode or while the metal housing portion of an implantable VNS stimulator is programmed or activated to function as a return (anode) electrode.

In a first general aspect, the disclosure provides a system for VNS, comprising: a VNS stimulator implanted in a subject and configured to transmit periodic electrical stimulation pulses to a vagus nerve of the subject using an electrode stimulation cuff comprising a circumferential array of electrodes; and a controller comprising a processor and memory, configured to cause the VNS stimulator to transmit the periodic electrical stimulation pulses to the vagus nerve by a) selecting an electrode in the circumferential array of electrodes as a cathode electrode; b) causing the stimulator to transmit an electrical stimulation pulse to the vagus nerve using the selected cathode electrode, while at least one other electrode in the circumferential array of electrodes is used as a return electrode or the metal housing on the VNS stimulator is functioning as an indifferent return anode; and c) repeating steps a) and b) at least once, wherein a different electrode in the circumferential array of electrodes is selected as the cathode electrode.

In some aspects, the circumferential array of electrodes comprises 2, 3, 4, 5, or 6 electrodes.

In some aspects, the circumferential array of electrodes comprises 4 arc-shaped or semicircular electrodes evenly spaced along a circumference of the electrode stimulation cuff, when the electrode stimulation cuff is viewed cross-sectionally.

In some aspects, each electrode in the circumferential array of electrodes is positioned along a circumference of the electrode stimulation cuff such that it is opposite to another electrode in the circumferential array of electrodes, when the electrode stimulation cuff is viewed cross-sectionally.

In some aspects, the controller is further configured to use all other electrodes in the circumferential array of electrodes, other than the selected cathode electrode, as return electrodes when performing step b).

In some aspects, when performing steps a) and b) above, a set of electrodes in the cuff are chosen concurrently to be cathodes, while the other electrodes in the cuff are off or inactive.

In some aspects, the controller is configured to cause the stimulator to transmit the electrical stimulation pulses: a) at different amplitudes, wherein each amplitude is independently determined for each selected cathode electrode; b) pulses at different amplitudes until a physiologic response is detected; and/or c) according to a periodic pattern.

In some aspects, the controller is configured to cause the stimulator to increase the amplitude, pulsewidth and/or frequency of the periodic electrical stimulation until an unwanted side effect is detected.

In some aspects, the controller is further configured to record a minimum amplitude, pulsewidth and/or frequency of stimulation provided by a selected electrode that caused the unwanted side effect.

In some aspects, the system further comprises one or more sensors configured to detect one or more physiological parameters of the subject being treated, and to determine whether the subject has experienced an unwanted side effect based on the detected one or more physiological parameters.

In some aspects, the one or more sensors comprise a heart rate sensor (e.g., an electrocardiogram (EKG) sensor, or a heart rate sensor that is an acoustic sensor such as a microphone), an electroencephalogram (EEG) sensor, and/or an electromyography (EMG) sensor.

In some aspects, the system described is directed to treating epilepsy by electrically stimulating the vagus nerve to suppress the onset of epileptic seizures or reducing the severity of epileptic seizures, while minimizing unwanted physiological side effects from the stimulation.

In a second general aspect, the disclosure provides a method for providing VNS, comprising: a) providing a VNS system comprising a stimulator implanted in a subject, an electrode stimulation cuff comprising a circumferential array of at least three electrodes, and a controller, comprising a processor and memory, configured to cause the stimulator to transmit the periodic electrical stimulation pulses to a nerve of the subject by i) selecting an electrode in the circumferential array of electrodes as a cathode electrode and one of (1) selecting at least one electrode as an anode electrode or (2) selecting the metal housing part of stimulator as the return anode, ii) causing the stimulator to transmit an electrical stimulation pulse to the selected cathode electrode, at a selected stimulus amplitude, stimulus pulsewidth and/or stimulus frequency, while at least one other electrode in the circumferential array of electrodes is used as a return electrode or the metal housing part of the stimulator is used as the return electrode; and iii) repeating steps i) and ii) at least once, by increasing at least one of the stimulus amplitude, pulsewidth and/or frequency until an unwanted side effect is detected, and then selecting a lower stimulus amplitude, pulsewidth, and/or frequency that eliminates or reduces the detected side effect, wherein the stimulus amplitude may be based on either constant current or constant voltage stimulus.

In some aspects, the selection of the cathode electrode is based on data obtained from monitoring the subject following prior electrical stimulation using the VNS system.

In some aspects, it will be understood that the general method for titrating which electrodes are selected as an active cathode or set of cathodes that will be used to apply therapeutic stimulation in a multi-electrode nerve cuff is not limited by the particular arrangements of the electrodes within the cuff. The general method of titration to select the active cathode electrode or electrodes, and the mode of stimulation, whether bipolar or unipolar, and the selection of the stimulus parameters, for example, the stimulus amplitude, can be applied to any cuff electrode that has at least 2 or more electrodes (for unipolar stimulation) and 3 or more electrodes (for bipolar stimulation).

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a VNS system for treating epilepsy using an implantable VNS stimulator coupled via a lead wire to a cuff electrode on the vagus nerve.

FIG. 2 is a is a perspective view of an electrode lead 118 that may be used as part of any of the VNS systems described herein (e.g., incorporated into the system shown in FIG. 1 as a substitute for the multi-cuff electrode shown in that exemplary aspect).

FIG. 3 is a cross-sectional view of the self-sizing nerve cuff electrode of the lead electrode shown in FIG. 2, shown in a furled state.

FIG. 4 is a diagram illustrating an exemplary embodiment of a VNS stimulator including a pulse generator and a controller configured to titrate one or more stimulation parameters, i.e., stimulus pulse width, stimulus frequency and stimulus amplitude.

FIGS. 5A and 5B are cross-sectional views illustrating two exemplary methods for nerve stimulation (e.g., VNS) according to the disclosure, using a cuff electrode.

FIG. 6 is a diagram of an exemplary EMG sensor unit and processing system used by a clinician in some embodiments.

FIG. 7 shows an example of a wireless EMG sensor system used by a clinician in some embodiments. In this diagram, multiple electrode cuffs are shown. However, in other aspects a single cuff with multiple electrodes positioned circumferentially around a target nerve (e.g., as shown by FIGS. 2-3) may be used.

FIG. 8 shows another example of the wireless EMG sensor system for automatically sending data directly to the VNS stimulator in some embodiments. In this diagram, multiple electrode cuffs are shown. However, in other aspects a single cuff with multiple electrodes positioned circumferentially around a target nerve (e.g., as shown by FIGS. 2-3) may be used.

FIG. 9 is an example diagram of an electrocardiogram (EKG) sensor including electrodes attached to a patient and a device for interfacing with a processing system. In this diagram, multiple electrode cuffs are shown. However, in other aspects a single cuff with multiple electrodes positioned circumferentially around a target nerve (e.g., as shown by FIGS. 2-3) may be used.

FIG. 10 is an example block diagram of a patient outfitted with different external sensors and an example sensor unit including a processing system according to an embodiment.

FIG. 11 is a conceptual diagram of an EEG sensor affixed to a patient's head and a device for interfacing with a processing system.

FIG. 12 is a conceptual flow diagram illustrating an exemplary method for providing VNS.

FIG. 13 is a conceptual flow diagram illustrating an exemplary method for identifying electrodes when providing VNS using systems according to the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

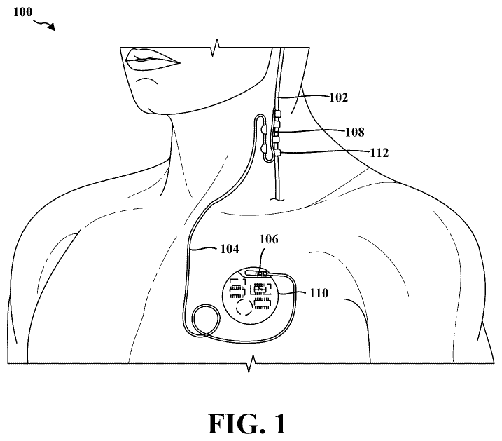

FIG. 1 is a diagram 100 illustrating an exemplary embodiment of a VNS system (e.g., for treating epilepsy or depression) using a VNS stimulator 110 implanted under the skin in the chest of a patient and coupled via a lead body 104 to a nerve cuff 108 having at least 3 electrodes placed circumferentially within the cuff when bipolar stimulation is use and at least 2 electrodes when unipolar or monopolar stimulation is used. The cuff 108 is placed around the vagus nerve 102. The cuff may have 2, 3, 4, 5, 6, 7 or more electrodes positioned circumferentially around the target nerve. The circumferential array of electrodes 112 may be located within the cuff that partially, substantially, or completely encircles a nerve. For example, a set of four arc-shaped or semi-circular electrodes may be placed within and evenly spaced along the circumference of a circular housing (e.g., to allow each electrode to stimulate a different quadrant of a cuffed nerve). In some embodiments, a single electrode or multiple electrodes within the cuff are used to deliver stimulation (function as cathode electrodes) while. concurrently, a different subset of one or more electrodes function as return electrode(s) (i.e., as an indifferent electrode or an anode). In some embodiments the VNS stimulator 110 may have a housing that is made at least partly of a metal such as titanium alloy which is electrically conductive and which can be selected or programmed to function as a return, anode, electrode (or indifferent electrode). A controller 220 of the stimulator 110 may be programmed to select the housing as inactive, in which case stimulation at the cuff can be operated in a bipolar stimulation mode—that is, at least one electrode in the cuff must be active as a cathode delivering electrical stimulation to a target nerve, while concurrently, at least one electrode in the cuff must be selected and operating as an anode. If the metal portion of the stimulator housing is programmed to function as a return, indifferent anode, then at least one of the electrodes in the cuff must be active and functioning as a cathode to deliver electrical stimulation to the target nerve. This latter mode of stimulation is called unipolar or monopolar stimulation. Not all electrodes may be active-some electrodes may remain inactive, meaning they do not function as either cathodes or anodes.

In some aspects, VNS stimulator 110 may also include a rechargeable battery which may be recharged inductively through the stimulator housing and through intact patient skin, i.e., transcutaneously. In some embodiments, the stimulator may include a single-use, primary-cell battery. VNS stimulator 110 may further include an outlet portion for enabling a connection between VNS stimulator 110 and lead body 104, which enables current flow to the vagus nerve 102 via the electrode stimulation cuff 108 and the one or more circumferential arrays of electrodes 112.

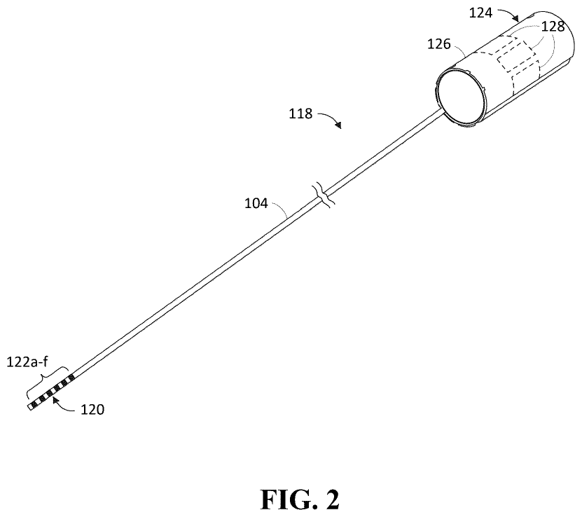

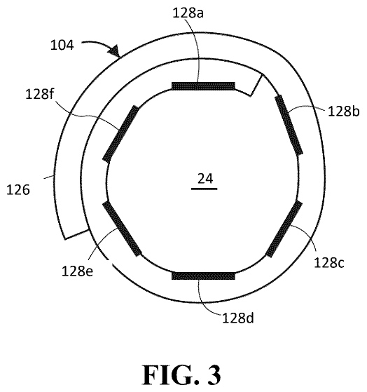

It should be expressly understood that the embodiment shown in FIG. 1 is a non-limiting example provided solely for illustrative purposes. In some aspects, electrodes are enclosed within the cuff). For example, in some embodiments the cuff geometry of a VNS system according to the disclosure may use a configuration as shown in FIGS. 2, and 4-6 of U.S. Pat. No. 10,967,178. Such embodiments are illustrated herein in the context of FIG. 2 and FIG. 3.

FIG. 2 is a is a perspective view of an electrode lead 118 that may be used as part of any of the VNS systems described herein. The proximal lead connector 120 comprises a linear array of connector contacts 122a-122f (in this case, six) for connecting to the connector receptacle of an implantable pulse generator when the proximal lead connector 120 is inserted into the connector receptacle. The nerve cuff electrode 124 comprises a nerve cuff body 126 that is capable of substantially or completely encircling the vagus nerve, and an array of electrode contacts 128a-128f (in this case, six) affixed to inside of the cuff body 126, such that when the cuff body 126 encircles the vagus nerve, the electrode contacts 128a-128f are in contact with the vagus nerve. The proximal lead connector 120 is coupled to the nerve cuff electrode 124 via an intervening lead body 104.

FIG. 3 is an end view of the nerve cuff electrode 124 of the electrode lead 104 shown in FIG. 2, in a furled state. The cuff body 126 is shown wrapped in a partially overlapped state (e.g., in a configuration as if it were wrapped circumferentially around the vagus nerve of a subject). In this furled state, the individual electrode contacts 128a-128f would form a circumferential array of electrodes 112 around the vagus nerve, allowing for unipolar or bipolar stimulation of the vagus nerve according to the methods described herein.

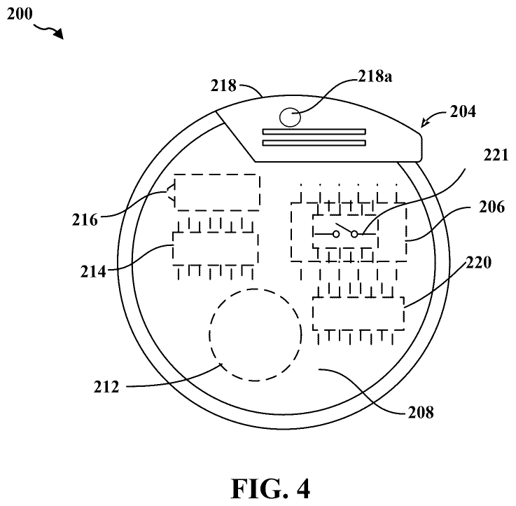

FIG. 4 is a diagram 200 illustrating an exemplary embodiment of a VNS stimulator 204 including a controller 220 with processing circuitry configured to control the transmission and/or parameters (e.g., the stimulus current amplitude, stimulus pulse width, stimulus frequency) of electrical stimulation provided by the VNS stimulator 204, as described in greater detail herein. For example, with bipolar stimulation, the controller 220 may be configured to classify one or more electrodes in the circumferential arrays of electrodes 112 of an electrode stimulation cuff as either a cathode electrode or as an anode electrode, and to activate stimulation of one or more of the cathode electrodes while the anode electrode(s) are concurrently used as a return. In some aspects, the classification of each electrode may be based on settings stored in memory 214 and/or provided by a clinician programmer device (e.g., via a wireless or wired connection as described herein).

The VNS stimulator 204 may include a rechargeable battery 212 that can be accessible for recharging inductively. In some embodiments the battery 212 may be a single-use, primary cell battery. The battery 212 of VNS stimulator 204 may be configured to supply power to a pulse generator 206, which may be programmed to generate a periodic electrical pulse having a set frequency and pulse width. The pulse generator can be activated and deactivated (the latter causing the stimulation pulses to the electrode or electrodes to be turned off) via a switch 221. The connections on the integrated circuits may be coupled together selectively via a small printed circuit board 208. In other embodiments, the VNS stimulator 204 may be implemented as an SoC on a die, or a packaged die.

VNS stimulator 204 may further include a transceiver/receiver 216. In some embodiments, transceiver 216 includes a wireless receiver configured to receive wireless signals, e.g., Bluetooth Low Energy, from a source external to the patient. In some embodiments, transceiver may further include a wireless transmitter, e.g., for providing feedback to a processor used in a clinician programmer device, or to an external sensor. In still other embodiments, the transceiver 216 may include a wire for receiving information from the vagus nerve or another part of the body. The wireless receiver in transceiver 216 may further be configured in some embodiments to receive instructions for the controller 220 to modify one or more stimulation parameters and/or titrate the stimulation pulse. Titrating a stimulation pulse, as defined herein means adjusting one or more of the stimulus parameters: pulse amplitude, pulse frequency, or pulsewidth to a value or values to yield a desired therapy. Titration as a general term may also include (a) selecting an electrode or electrodes as a cathode, and an electrode as an anode (for bipolar stimulation) or (b) selecting an electrode or electrodes as a cathode, and the metal portion of the VNS stimulator housing as the indifferent, return anode (for unipolar stimulation). In the case of treating epilepsy, successfully titrating the stimuli delivered to the vagus nerve will prevent seizures before their onset or, if seizures occur, will reduce the severity and duration of the seizure. Titration of the stimulation pulses should be performed to find the stimulus parameters that stimulates the target nerve fibers that provides efficacious response, for example, with epilepsy to prevent or reduce the severity or number of occurrences of epileptic seizures. In another treatment example, titrating the stimulation pulses to treat chronic depression, the stimulus parameters are selected and optimized to reduce the severity of depression, if not eliminate depression. The wireless transceiver 216 may further be configured to receive information including events recorded or detected by one or more external sensors. When acting as a transmitter, the transceiver 216 can provide feedback signals to external sources using data generated by controller 220. In some embodiments, the information received from the wireless receiver/transceiver 216 may be provided to a memory 214. The controller 220 may access the memory 214 to receive and process instructions to deliver electrical pulses to one or more electrode(s) of each circumferential arrays of electrodes 112, or to temporarily deactivate the pulse generator 206.

While various functions of the VNS stimulator 204 have been shown, it will be appreciated by those skilled in the art upon review of this disclosure that different architectures may be used. For example, the controller 220 may include more than one integrated circuit, or it may include a separate module coupled to the VNS stimulator. The controller 220 may further include one or more general purpose processors, RISC processors, or other types of processors. The controller 220 may in some embodiments include dedicated hardware. For example, the controller 220 may be any one or more of a digital signal processor (DSP), a field programmable gate array (FPGA), an application-specific integrated circuit (ASIC), or a combination of digital logic devices.

The memory 214 may include any suitable memory, such as a combination of volatile and non-volatile memory, dynamic random access memory (DRAM), static random access memory (SRAM), read only memory, flash or other solid state memory, or the like. Other types of memory are possible. The battery 212 may supply the memory 214 with power as necessary. Non-volatile memory within memory 214 may be used to store critical settings to enable system reset, for example. Stimulus pulse frequency and pulse width values may be stored in the memory. In some embodiments, the memory may be accessible and programmable as noted above via the controller 220, or via data or instructions received at via wireless receiver 216. The memory 214 may also include firmware for use by the controller, or other program information for automatically titrating one or more of stimulus pulse width, pulse frequency and pulse amplitude.

Stimulation parameters applied by the controller 220 may be based, in part or in whole, on information received from one or more sensors (e.g., EEG, EKG or EMG sensors) configured to detect one or more physical parameters of the subject being treated, or based upon input from the subject (e.g., a report that current or prior settings resulted in undesirable side effects). In some aspects, a subject may be monitored over time while VNS is provided using a system or method described herein, and this information may be used to determine whether changes to one or more stimulation parameters are needed. For example, monitoring may be used to determine which electrode(s) in each circumferential array of electrodes 112 should be used to deliver stimulation (i.e., as cathode electrodes), and/or to determine an appropriate amplitude level for one or more such electrodes, in order to minimize undesirable side effects. Over time, an acceptable or generally optimal configuration of stimulation parameters may thus be identified for the subject. However, it will be appreciated that in other applications, acceptable parameters may be defined differently. For example, a clinician may observe that while a lower amplitude is effective in reducing seizures, a slightly higher amplitude may be more effective and may reduce seizures, without the slightly higher amplitude producing unwanted side effects (or minimal side effects).

In short, the “acceptable” (e.g., optimal) configuration and/or amplitude of the electrical stimulus pulses may be different for each subject. In some embodiments, an acceptable amplitude may fall within a range of amplitudes. Generally, smaller stimulus amplitudes may advantageously increase the time needed between recharges of the battery 212 in the VNS stimulator 204. Other considerations may dictate the need for different amplitudes (e.g., substantially increased efficacy in reducing seizures, or improving depression in cases where depression is indicated). The welfare of the patient remains a priority, and it is often beneficial to have a lower amplitude, as a lower current may be deemed less invasive in many cases. Shorter pulse widths and lower stimulus frequencies may have more or less side effects for a given stimulus amplitude.

It will be appreciated that VNS stimulator 204 need not be circular or elliptical in nature, and may take on different shapes based on different design considerations and patient needs. More generally, the components identified in the various figures may take on different geometries than those shown.

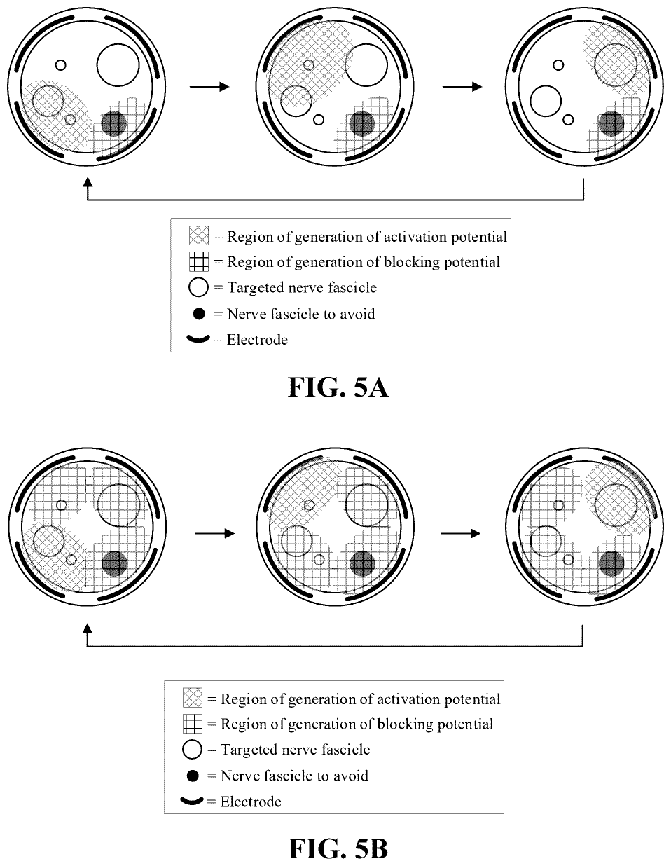

FIGS. 5A and 5B show a cross-sectional view of the nerve cuff having four electrodes in a circumferential arrangement within the cuff and around the vagus nerve bundle. The vagus nerve bundle has different nerve fascicles or nerve fibers having different diameters. Other embodiments of the nerve cuff can have circumferential array of electrodes comprising a plurality of electrodes (e.g., 2, 3, 4, 5, 6, 7 or 8) positioned along a circumference of the inside of the cuff body 324, when viewed cross-sectionally. Each electrode may be, e.g., semicircular or arc-shaped when viewed cross-sectionally. In some aspects, the electrodes may be evenly spaced along a circumference of the cuff body 324, when viewed cross-sectionally.

The concepts disclosed herein may be used following the initial implantation procedure. In addition, even after use of the VNS stimulator commences, an assessment is typically performed periodically to re-assess the effectiveness of the VNS therapy. Particularly where a sub-optimal or less than expected efficacy is observed, the principles of this disclosure can be applied after each periodic assessment, for example, in order to reestablish the acceptable stimulus parameters, including amplitude of the stimulus pulse or to identify more acceptable stimulus parameters, including amplitude with the potential prospects of yielding greater results.

FIGS. 5A and 5B illustrate two exemplary methods for providing nerve stimulation (e.g., VNS) according to the disclosure. These example shows cross-sectional views of a vagus nerve encircled by a nerve cuff comprising a circumferential array of electrodes according to the disclosure, as stimulation is applied according to two different patterns referred to herein as “Method A” (shown in FIG. 5A) and “Method B” (shown in FIG. 5B), respectively. In these examples, the circumferential array of electrodes comprises four arc-shaped electrodes as viewed in the cross-section, evenly spaced along the circumference of a circular cuff, allowing each electrode to focus stimulation on a different quadrant of the encircled nerve. In Method A, the lower-left quadrant electrode is selected as the initial cathode electrode and the lower-right quadrant has been concurrently selected as the anode (return) electrode. The upper-left quadrant selected as the next cathode electrode, followed by the upper-right quadrant electrode. As illustrated by Method A, electrical stimulation may be provided to the three cathode electrodes in sequential order, following a “round robin” approach, while the anode electrode (lower-right quadrant) is maintained as a return. In contrast, in Method B (shown in FIG. 5B), stimulation proceeds according to the same pattern, but in each case all of the remaining electrodes in the circumferential array of electrodes (other than the one selected as the cathode electrode) are used as the anode (return) electrode. The stimulation process of Methods A or Method B may be repeated as needed, and in particular may be advantageously used to generate action potentials on many type-B fibers without generating undesirable side effects via motor-neuron activation, such as laryngeal motor effects and cardiac effect. Such methods are particularly useful for stimulating the cervical vagus nerve.

The specific stimulation pattern to use with bipolar stimulation, i.e., the choice of electrodes within the cuff acting as a cathode or as an anode or anodes, and the parameters of stimulation (e.g., pulse amplitude, pulsewidth, and frequency) may be selected by a medical practitioner or based on prior studies of the subject being treated. For example, stimulation delivery may be started with a pulse amplitude at 0.1 mA while monitoring for side effects. After determining if this causes side effects, the stimulation amplitude may be increased, e.g., by 0.1 mA, and more stimulation pulses may be delivered while monitoring for side effects. In some aspects, the amplitude may be raised continuously or periodically until one or more side effects are observed or a stimulation current is reached wherein it is expected that all type-A fibers have been recruited on the side (or quadrant) of the nerve closest to the stimulating cathode. This process may be repeated for each of the remaining electrodes in the circumferential array of electrodes, allowing a medical practitioner to select which electrode to use as the cathode and which electrode to use an anode or, in some cases, which electrodes to use as anodes in a customized treatment plan for each subject. Such plans may comprise the selection of one or more electrodes in the circumferential array of electrodes to be used as cathode electrodes and/or parameters of stimulus pulse delivered by each selected electrode (e.g., the amplitude, pulse width or frequency of stimulation transmitted to each electrode). Some electrode or electrodes within the cuff may not be selected and will not function as either a cathode or anode.

In some aspects, in bipolar stimulation mode, electrodes that cause side effects may be used as the return electrode(s) or anodes. In the case of exemplary Method A, these electrode(s) may be used as the anodes and stimulation pulses may be alternatively sent to each of the other electrodes in the circumferential array of electrodes (e.g., following the “round robin” approach described above). In the case of Method B, stimulation is provided using each electrode that was found not to produce side effects as a cathode while all other electrodes function as a return electrode. This configuration may help to reduce the number of side-effect producing nerve fibers that are activated with each stimulation pulse.

The foregoing discussion of the method of titrating which cathode and anode within the cuff to select as the active electrodes, and for determining them based on best efficacy with the least amount of side effects, is based on bipolar stimulation. However, it will be understood that a similar titration strategy can be used with unipolar stimulation is employed, where the metal portion of the stimulator housing is used as an indifferent, return, anode. In such a unipolar stimulation, each electrode within the cuff may be selected successively as a cathode, while the metal portion of the VNS stimulator housing is operating as the return anode. Cycling through each possible cathode electrodes, or sets of cathodes, the stimulation parameters, e.g., the stimulus current amplitude, will be adjusted upwards and then downwards until unwanted physiological side effects are minimized, while maximizing the efficacy of the stimulation. At the end of this titration process, while in a unipolar stimulation mode, the optimal cathode or set of cathodes will be selected with the optimal stimulus parameters, e.g., stimulus amplitude, at a pre-selected stimulus pulsewidth and pulse frequency.

With respect to treating epilepsy, various temporary measurement devices and sensors can be used to identify and maximize stimulation efficacy and also minimize unwanted side effects, by using various types of heart rate sensors, e.g. EKG, sound detecting microphone sensors, IMUs to detect heart rate using motion detection, which can be used to detect ictal tachycardia (too fast heart beat as indicator for the onset of epileptic seizure) or bradycardia (as indicator of an unwanted cardiac side effect-too slow heart beat), EMGs to detect unwanted muscle contraction in the head and neck areas, EEGs to detect possible onset of seizures or indication of suppression of seizures as an indicator of vagus nerve stimulation efficaciousness in treating seizures. In some embodiments, a plurality of electrodes may be chosen as active cathodes, while the metal portion of the VNS stimulator housing functions as the indifferent, return, anode. The same iterative procedure is carried with each selected combination of a set of active cathodes to find the best set that provides the most efficacious treatment with the least amount of unwanted side effects.

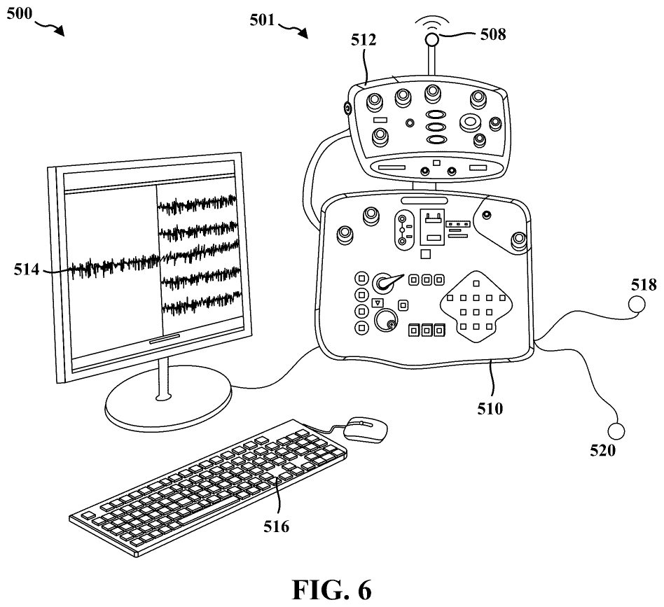

FIG. 6 is a diagram 500 of an exemplary electromyography (EMG) unit 501 used by a clinician in some embodiments (e.g., to determine whether a current treatment plan generates undesirable side effects such as laryngeal muscle recruitment as evidenced by electromyography activation). Laryngeal muscle recruitment is a common unwanted side effect of stimulating the cervical vagal nerve. The EMG unit 501 may include a body 510 in which a processing system can be housed, and a controller 512 for tuning and manipulating the various options used in EMG measurement procedures. In some embodiments, the EMG may include wires for use with the patient including one or more surface or percutaneous needle sensing electrodes 518, 520. As described below, in other embodiments, the EMG electrodes may be wireless, or they may be coupled to a Bluetooth or other wireless device. Information about events detected during EMG monitoring can be received by the EMG unit via antenna 508 in those embodiments.

A user interface with a keyboard 516 and mouse 517 can be used by a clinician to observe events 514 on the screen detected during an EMG monitoring session. In various aspects of the disclosure, the detected events can be used by the clinician concurrently with titration of the stimulus parameters, including stimulus pulse amplitude using the VNS stimulator and also selection of an active electrode or electrodes.

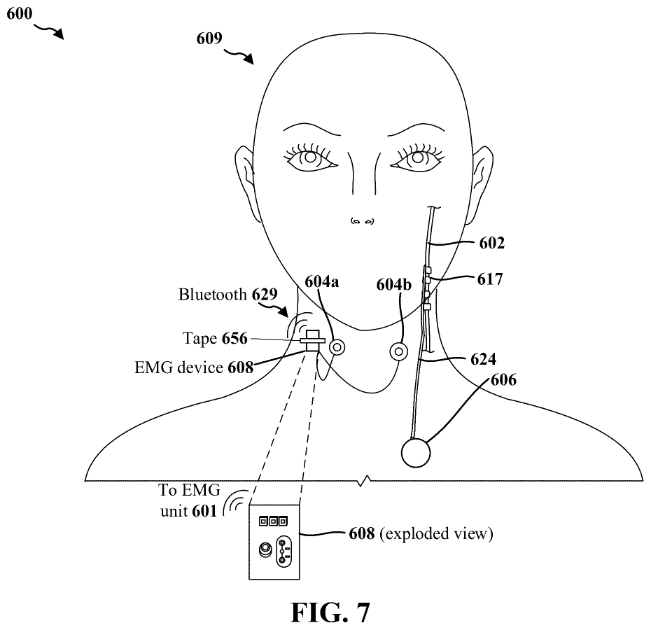

To this end, FIG. 7 shows an example diagram 600 of an external wireless EMG sensor system used by a clinician in some embodiments. A patient 609 is shown along with an exemplary front view of the patient's larynx. It will be appreciated that the larynx is not exposed via surgery (i.e., it is underneath the skin surface), but the patient is awake and conscious and the larynx is being shown through the patient's skin for clarity. In an embodiment, the patient 609 has a VNS stimulator 606 implanted under the skin in the chest area. As before, the VNS stimulator is coupled via lead wire 624 and electrode cuff 617 to vagus nerve 602. It will be appreciated that the structures in FIG. 7 are not necessarily drawn to scale, but are instead shown for clarity.

An EMG sensor may be placed on either side of the larynx. The EMG sensor includes a pair of surface or percutaneous needle electrodes 604a and 604b coupled to a small wireless transceiver 608. The wireless transceiver 608 may be affixed using tape 656 or some other adhesive means or otherwise externally affixed to a region on the skin adjacent the larynx, such as a throat area of the patient 609. An exploded view of the transceiver 608 is shown. Coupled to transceiver 608 are the two surface electrodes 604a and 604b attached over the skin to each side of the larynx. In other configurations, a single electrode or multiple electrode may be used across the larynx or different areas of the throat as determined in the discretion of the clinician.

The procedure as described herein is anticipated to be used following the initial system implantation and the periodic post implantation periods (conducted annually or otherwise). Measurements from the EMG sensor 608 can be transmitted wirelessly, e.g., via Bluetooth Low Energy to minimize interference, to the EMG unit 501 to enable the clinician to observe the EMG results via monitor 514.

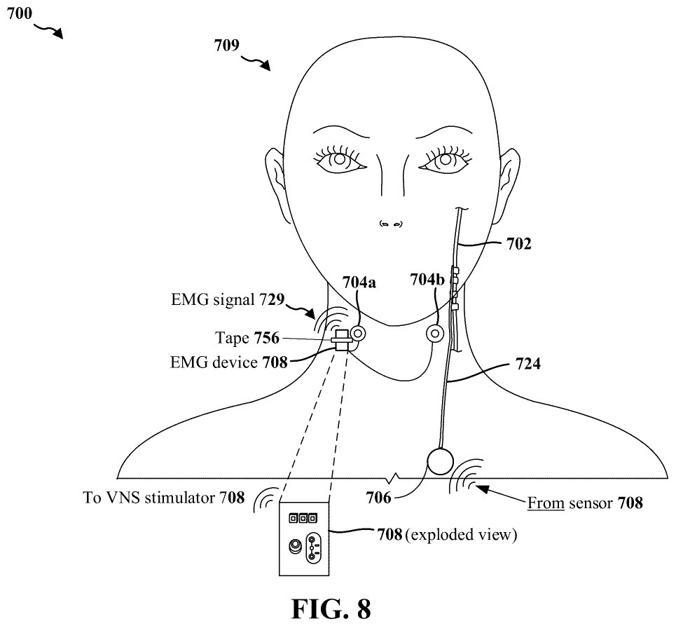

FIG. 8 shows another example of the wireless EMG sensor system 700 for sending data directly to the VNS stimulator in some embodiments. As before, a clinician may configure a patient 709 with an electrode 704a and 704b on each side of the throat. The clinician may use tape 756 or other mechanism to affix compact EMG device on the neck area. VNS stimulator 706 may include a wireless receiver as shown in the text “from sensor 708” in FIG. 8. Lead wire 724 is connected to the vagus nerve 702. This embodiment includes further automation. Instead of transmitting the signal to the EMG unit 501 as in the embodiment described with reference to FIG. 7, the EMG signal 729 of FIG. 8 can be additionally or alternatively transmitted by the EMG device 708 to the wireless (or wired) receiver 216 on the VNS stimulator 204 of FIG. 4 (or in this example, to the VNS stimulator 706). In some embodiments, the signal 708 can be provided to the EMG unit 501 where the readings are also made available to the clinician as well as the VNS stimulator 706. With the initial need to select potential cathode and anode electrodes from the circumferential array of electrodes, as well as the need for initial and subsequent titrations with a large number of combinations of stimulus parameters in order to identify an optimal treatment plan, the use of objective EMG readings facilitates the optimization process from the perspective of automaticity, time efficiency, and accuracy.

In various embodiments, additional external sensors can be used concurrently with the EMG sensor to identify other events relevant to acceptable amplitude levels. Certain such embodiments are described further below. Referring back to FIG. 7, when the EMG sensor as described above is affixed externally on the patient to the larynx region of the throat, the EMG sensor (e.g., transceiver 608 and sensing surface or percutaneous needle electrodes 604a-b) may be used to detect muscle movements caused by in this case, by unwanted efferent stimulation of nerve fibers in the vagus nerve 602. A laryngeal muscle contraction may be detected, or other events indicative of side effects or unwanted phenomena. It would be desirable to avoid having any of the vagal stimulation pulses reach the laryngeal branch of the vagus nerve 602 and cause unwanted laryngeal muscle contraction side effect. In the case of stimulating the vagus nerve to treat epilepsy, for example, the nerve stimulation should largely target Type B afferent nerves to transmit desired signals to the brain in order to suppress the onset of or reduce the severity of epileptic seizures, while avoiding or reducing unwanted motor nerve stimulation to the muscles, such as laryngeal muscles and to avoid, to the extent possible, other unwanted physiological effects mediated by various nerve fibers in the vagus nerve. For example, the cervical vagus nerve is connected to organs such as the heart and to the gut.

The clinician can select the position of the appropriate surface or percutaneous sensing electrodes 604a-b that detect electrical activity emanating from muscles when they are activated to contract. The clinician or the controller 220, in the case of full automation, can use the readings from the EMG sensor to slowly titrate up or, as needed, step down the pulse amplitude of the VNS stimulator 606 over time, e.g., when optimizing a treatment plan for the subject. For example, the clinician can use the monitor 514 to make readings based on the unwanted physiological side effects, e.g., unwanted muscle contractions detected from the EMG sensor. As another example, once unwanted muscle stimulation {Note: EMG does not detect nerve electrical conduction directly-only indirectly by sensing the resulting muscle contraction electrical activity} is detected as indicated by EMG readings, or other bodily events are detected relevant to identifying an acceptable stimulation amplitude (or in some cases, pulsewidth or frequency) which, to the extent possible, reduces or eliminates the unwanted muscle contracts as described below, the clinician or controller may stop titrating the stimulus amplitude (or stimulus pulsewidth or stimulus frequency), via the controller or other conventional methods used for titrating the stimulus parameters. In some embodiments, for certain side effects identified by the EMG sensor (e.g., contraction of the larynx), the controller 220 or clinician may determine that a downward stimulation titration is necessary.

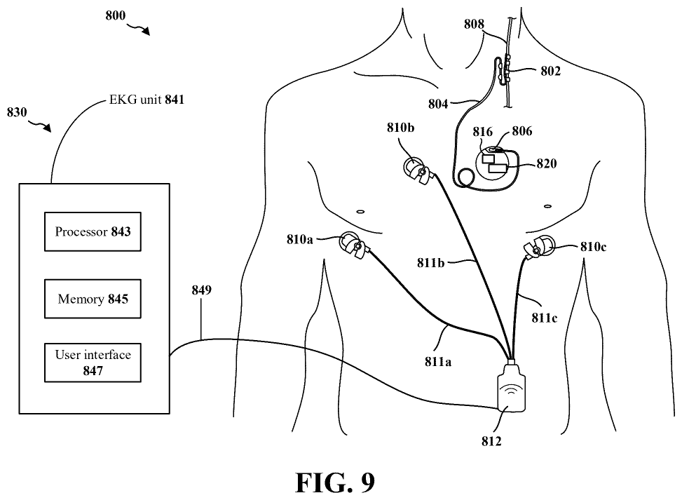

FIG. 9 is an illustration 800 of an embodiment of a patient equipped with an external EKG sensor 830 and an implanted VNS stimulator 806. Bradycardia (too slow heart beat) and tachycardia (heart beat which is too fast) is a common unwanted side effect of stimulating the vagus nerve. Wires may run from external compact device 812 to an EKG monitor unit 841 analogous to the EMG unit 501. In the illustrated embodiment, the compact device includes a processor and an interface for coupling to EKG lead wires 811a, 811b, and 811c. The EKG lead wires may terminate in respective lead wire terminals for attaching to corresponding lead wires 811a, 811b and 811c, the latter of which are taped or otherwise affixed on selected regions of the patient. In various embodiments, different numbers of lead wires and electrodes may be used (e.g., five electrodes, etc.). The compact device 812 may include one or more wires 849 directly attached to the EKG unit 841 for sending data from the surface electrodes 810a-c to the EKG unit 841. In the embodiment shown, EKG unit 841 may be a larger dedicated unit for receiving and measuring detected physical events at electrodes 810a-c. The EKG unit 841 may include a processor for running instructions that the controller 841 stores in the memory 845. The memory 845 may also store the detected events for evaluation by the processor 843 or by another device. In addition, a user interface 847, which may include a touch screen, keyboard, mouse, and the like, may be part of EKG unit 841.

In other embodiments, the wire 849 may be absent and the compact device 812 may include a wireless transmitter for sending wireless transmissions to an EKG unit pursuant to any number of wireless protocols.

VNS stimulator 806 may be implanted in the patient via lead wire 804 to the vagus nerve 808. The signals may be read by the controller 820 for use in titrating the stimulus parameters, including stimulus amplitude for one or more electrode pairs within cuff 802 for bipolar stimulation. Alternatively, or in addition, the compact device 812 may send the signals from the lead wires 811a-c to wireless transceiver 816 on VNS stimulator 806 for providing the signals directly to the controller 820 on VNS stimulator 806. More generally, an EKG is an external, sensor-based system used for recording the electrical signals in the heart. EKGs may be used to detect abnormal heart rhythms (arrhythmias), evidence of blocked arteries, pacemaker analyses and other events relevant to functioning of the heart.

In another aspect of the disclosure, physical events from an EKG are measured and provided to the clinician and/or a controller via user interface 847 or via controller 820 of the VNS stimulator 806. The clinician may use a monitor as part of the user interface 847 to view the signals from the electrodes. During titration of stimulus parameters, e.g., stimulus amplitude for the vagus nerve 808 and the selection of cathode and return electrodes from the circumferential array of electrodes following the initial fitting, the EKG may be monitored to detect ictal tachycardia events as a surrogate for seizure detection. It is known that the occurrence of seizures is associated with occurrence of ictal tachycardia. The titration of stimulus parameters is performed to optimize (a) the selection of cathodes and anodes within the cuff and (b) selection of stimulus parameters which selections (a) and (b), to the maximum extent possible, eliminate or reduce unwanted side effects to cardiac function such as bradycardia-a too slow heartbeat, while still delivering efficacious stimulation therapy for conditions such as epilepsy, depression, and other conditions, that are amenable to treatment with vagus nerve stimulation. It is known that stimulation to vagus nerve may cause unwanted bradycardia. In various embodiments, the EKG sensor may be used concurrently with other sensors, or on its own.

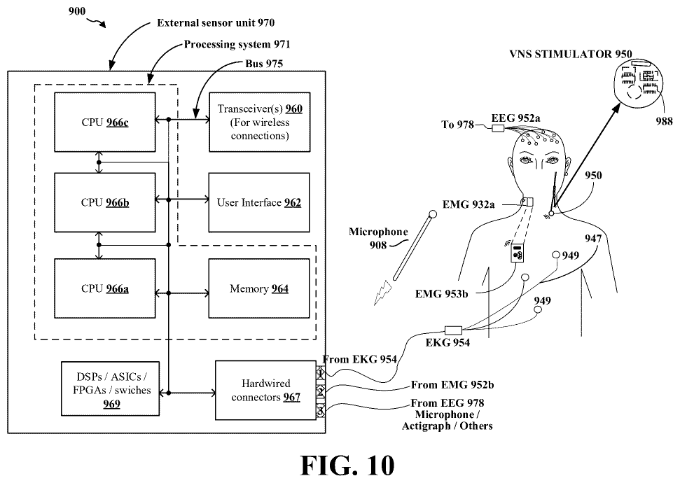

In other embodiments, a microphone (FIG. 10) may be used as a sensor to monitor heart rate and detect ictal tachycardia events by detecting heart sounds In still other embodiments, the stimulus parameters, e.g., stimulus amplitude may either be increased from a minimum value, or decreased from the maximum value previously determined using the larynx EMG sensor as described in embodiments above. In yet other embodiments, a wireless microphone with an onboard inertial measurement unit (IMU), or a Micro-Electro Mechanical Systems (MEMS) microphone may be used to detect various unwanted physical events including cough, which is one of the common side effects of VNS stimulation. Advantageously, certain external sensors such as these can be used in an outpatient setting, including at the patient's home.

These embodiments rely on the fact that an increase in heart rhythm is common during a seizure. One type of epileptic seizure is known as ictal tachycardia, in which the subject's heart rate increase of more than ten (10) beats per minute of above the baseline. Epileptic seizures can lead to changes in autonomic function affecting the nervous systems. Changes in cardiac signals are potential biomarkers that may provide an extra-cerebral indicator of seizure onset in some patients. As a result, EKG sensors can assist the controller 220/820 in detecting cardiac events, including their number and magnitude, that may be associated with an upcoming seizure. Accelerations of cardiac events can be measured, thereby allowing the early detection of arrythmias (such as ventricular fibrillation) that may cause death. It is generally reported that significant heart rate changes are associated with a large number of patients that experience epilepsy. The EKG sensor and/or a microphone or stethoscope can be used to detect these changes in heart rate, including ictal tachycardia events. It will be understood that, as with an EKG sensor, the other sensors may be used as heart rate sensors, e.g., a microphone/acoustic sensor to detect heart sounds and thereby heart rates, an IMU for detecting heart motions to detect heart rates, which may be used to detect ictal tachycardia as a surrogate for epileptic seizures and also for detecting unwanted cardiac side effects, such as bradycardia or tachycardia that is not associated with seizures.

In various embodiments, each change in stimulus amplitude made based on the number of ictal tachycardia events or other heat rate phenomena can be monitored for a relatively long duration, for example, about one week. The controller 220 (FIG. 4) may store these events in memory. The clinician and/or controller can compare the average number of ictal tachycardia events, e.g., over the week or other duration of time. If the change in the number of ictal tachycardia events is significant, such as above some threshold, the controller 820 or clinician may increase the pulse amplitude again to attempt to increase the efficacy of the VNS stimulator. This comparison may be repeated for two or more periods of time until no further reduction in ictal tachycardias is observed.

In some embodiments, the above information can be maintained and processed using a processing system external to the patient. Thereafter, relevant information about the detected EKG events can be downloaded to the controller. In some embodiments where the data is processed externally or by a clinician, an external processing system may send an instruction to the VNS stimulator to titrate the stimulus parameters, e.g., stimulus amplitude. In still other aspects, while monitoring for ictal tachycardias, the EKG sensor can also monitor, and a processor can maintain a count of, the total number of bradycardias. A bradycardia is a slower than normal heart rate, such as less than sixty beats per minute. A bradycardia can stop the brain or other vital organs from receiving enough oxygen, which can result in various side effects and potentially dangerous symptoms. An increase in bradycardias can be an undesirable side effect of high stimulation. Accordingly, in various embodiments, where the EKG or other external sensor identifies some threshold number of bradycardias, the periodic increase in stimulus amplitude may be halted.

Other physical events can be relevant to an acceptable stimulus pulse amplitude. An ACTi graph is a type of accelerometer that may measure sleep parameters and motor events over the course of days or weeks. These events may be relevant to titrating a stimulus amplitude. Prone position events such as acute respiratory distress may be measured by a ventilator. Still other heart rate events may also be measured, such as low heart rate events. Heart Rate Variability (HRV), which can also be measured by an EKG, can be measured so that the efficiency of VNS therapy on patients with epilepsy who have bradycardia or normal heart rate can be compared to patients who have ictal tachycardia.

Another aspect of the disclosure involves patient feedback through a user interface coupled to or included within an electronic device, such as a specialized medical device or a general purpose computer (PC, mobile phone, laptop, etc.). Some aspect of stimulation may be increased autonomously. This stimulation may occur in some instances without the need for patient notification. In other instances, a mechanism for the patient to provide feedback may be made available through the patient's controller 220 (FIG. 4), which may include an application on a PC or mobile phone, or other device. In various embodiments, this mechanism may include a button, switch or other selection means included on the patient controller. The patient may press the button or select the switch if the patient or clinician (caregiver, physician, and the like) notices an event from the patient. An event may include side effects. Common such side effects may include a voice change, a sore throat, heart palpitations, difficulty swallowing, paresthesia, insomnia, shortness of breath, and the like. In some embodiments, the patient controller may include queries for the client to answer (such as in the computer application on the patient controller) to enable the patient controller to determine the nature of the patient's problem. In other embodiments, the application may present a periodic survey to prompt the patient/caregiver to assess whether side effects due to the VNS stimulation have occurred. The feedback provided by the patient as a result of the survey may indicate that the patient has experienced no side effects and is comfortable. This information may also be used to prompt the next change in titration of the pulse amplitude.

The ability to view long term data or trends of the patient's tolerance to each adjustment to the amplitude can help determine whether one or more side effects may be decreasing over time. Some configurations may involve a wireless microphone (908) with an onboard inertial measurement unit (IMU) or a Micro-Electro Mechanical Systems (MEMS) microphone, may be used to detect the patient's cough, which is one of the common side effects of VNS stimulation, and record the data in the patient controller.

FIG. 10 is an example block diagram 900 of a patient outfitted with different external sensors and an example sensor unit including a processing system according to an embodiment. FIG. 10 includes an external sensor unit 970 for assessing one or more of EKG, EEG, EMG sensor data, and other sensor data such as auditory and inertial external sensor data. An EEG recording can help provide long term data correlating whether a particular cathode electrode selection and stimulation parameters are effective in reducing the number of occurrences of seizures and the severity of seizure. It should be understood that the unit 970 in practice may instead be multiple units that specialize in processing data for different respective sensors. Thus, external sensor data may in practice include an EEG unit, an EKG unit, and EMG unit, and so on. In various embodiments, unit 970 may include a general purpose processing system such as a personal computer, a server, or the like. Other embodiments may include multiple units corresponding to multiple sensors along with a central processing system housed in one of the units (or another unit) for consolidating and analyzing the data.

A patient may be adorned with EEG sensors 952a. The sensors may include lead wires that terminate at a compact EEG sensor 952a. The compact EEG sensor 952a may be connected via hardwire as shown in hardwired connector 967 at port 3. In some embodiments, the compact EEG sensor 952a may instead transmit its data to the unit 970 using a suitable wireless technology. In addition, in some embodiments, either unit 970 or compact EEG sensor 952a may be configured to transmit data to the VNS pulse stimulator 950 for use by controller 988, wirelessly or otherwise.

The patient may also wear an EMG unit 953a (exploded view in 953b) along with EMG electrodes (not shown) to enable a clinician to perform EMG tests. The EMG unit 953b may be hardwired to hardwired connector 967 at port 2 to provide the external sensor data to external sensor unit 970 (e.g., an EMG unit). In various embodiments, the EMG unit 953a may instead transmit the sensor data using Bluetooth Low Energy, or another wireless technology.

A microphone 908 and other auditory sensors may be used to determine cardiac events. The information may be provided to a user interface 962 (e.g., where unit 970 is a personal computer or specialized external unit). In some embodiments, microphone 908 may include a wireless connection to transmit information wirelessly to the unit 870 or the VNS stimulator 950.

Referring still to FIG. 10, an expanded view of an implantable VNS stimulator 950 is shown. Controller 988 on the VNS stimulator 950 may in some embodiments receive data or instructions, wirelessly to external sensors and/or unit 970 or through a temporary wired connection, that connects the implanted stimulator to external sensors and/or unit 970. VNS stimulator 950 can use these data or instructions to titrate the amplitude of the electrodes on the pulse generator in some embodiments.

The external sensor unit 970 in FIG. 10 may further include a transceiver 960 to effect one or more wireless connections, whether to or from the sensors as described above, or to or from controller 988. Where unit 970 includes a plurality of units specific to different sensors, transceiver 960 may be used to obtain data regarding physical events to titrate the selection of stimulus parameters, e.g., stimulus amplitude, and selection of an active cathode or cathodes and active anode or anodes, in bipolar stimulation, and electrodes that are not used or inactive within the cuff during the optimization process. In other embodiments, the stimulus amplitude may be titrated based on the external sensor unit 970.

The patient may also, concurrently or at a different time, be outfitted with EKG electrodes 949. EKG electrodes may provide data regarding heart-related events through lead wires 947 to a compact EKG device 954. In some embodiments, EKG device 954 may include wires to the external sensor unit 970 (e.g., an EKG unit) at port 1, as shown in FIG. 10. EKG device 954 in some embodiments may be configured as a wireless device, providing the EKG sensor data to the external sensor unit 970 using transceiver 960. Further, in various embodiments, either EKG device 954 or external sensor unit 970 may transmit EKG sensor data or instructions relating thereto to the VNS stimulator for use by controller 988.

In various embodiments discussed above with reference to FIG. 6 and FIG. 7, the compact device affixed to the patient's neck may automatically transmit the detected information to a unit, such as transceiver 220 on the VNS stimulator 218. In other embodiments, the transceiver (220) may also send the information to a unit like unit 970 to enable the clinician to review the results. Unit 970 may also include a bus system 975 to connect all the components together. A memory 964 may be used to store data corresponding to the outputs of one or more sensors. The memory may include one or more hard drives (solid state or magnetic), DRAM, SRAM, programmable memory such as PROMs, EPROMS, EEPROMs, flash memory, and/or other volatile and nonvolatile means of storage. In addition, various types of hardware components 969 (e.g., DSPs, ASICs, FPGAs, switches and other devices may be used, and in some cases, included at least in part as a portion of the processing system.

Processing system 971 may include one or more CPUs 966a-c and memory 964. The processing system in some embodiments may be configured to evaluate the received sensor data including (i) specialized EMG data if the external unit 970 is an EMG sensor, for example, or (ii) multiple physical events from multiple sensors, if the external unit 970 is configured to include a sophisticated processing system with code to recognize and evaluate different types of sensor data.

In various embodiments, the processing system 971 can apply weights and significances to these events and can determine, using the consolidated sensor data, an appropriate titration schedule. The processing system 971 is shown to include CPUs 966a-c, but in other embodiments the processing system 971 may perform one or more functions, at least in part, using dedicated hardware 969. In various embodiments, the processing system uses the hardwired connectors 967 or the transceiver to transmit data to, or receive data from, one or more sensor as well as the VNS stimulator 950. Using multiple external sensors can provide significant advantages in identifying an optimal set of pulse amplitudes for a multi cathode cuff, for example. The combination of different such measurements may in some cases be reinforcing, and the probability of success in titrating the stimulus parameters and, particularly, stimulus amplitude based on a combination of physical events may provide a maximally acceptable amplitude at each electrode/cathode for reducing seizures or depression, etc., without causing undesirable side effects (e.g., by avoiding or minimizing recruitment of type-A nerve fibers).

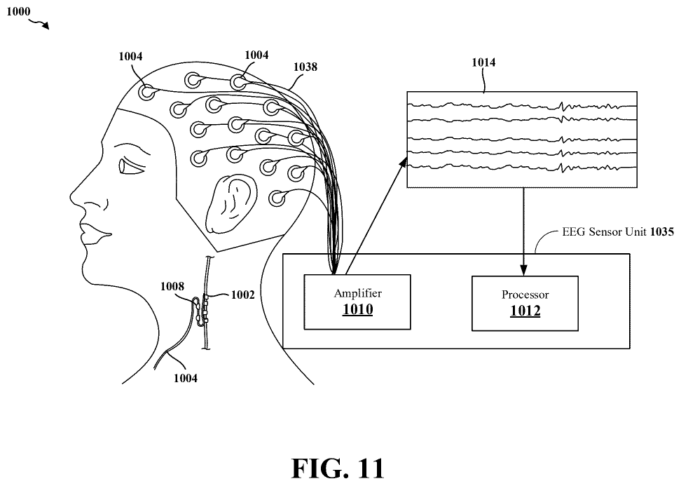

FIG. 11 is a conceptual diagram of an EEG sensor affixed to a patient's head and a device for interfacing with a processing system. An EEG, or electroencephalogram, measures electrical activity, including abnormal activity, in the brain. The clinician may place a flexible cap or connected assembly of small electrodes 1004 (here, conducting discs) on the scalp. The signals from the brain flow through the lead wires 1038 to an EEG sensor unit 1035 (similar to the EKG and EMG units). The sensor unit 1035 includes amplifier 1010. Because the electrical signals from the brain are very small, the amplifier 1010 can be used to boost the signal strength to a level that processor 1012 can utilize. The EEG sensor unit 1035 can include additional components, including memory, dedicated hardware for performing specific tasks, and a user interface panel. The user interface may include screen 1014 in which the electrical activity can be viewed.

In measuring brain activity, the EEG can also recognize improvements and therapeutic effects as brain activity stabilizes (e.g., as a result of the pulses generated by VNS stimulator 204). For example, at the outset of titration therapy, the EEG can make measurements as a baseline, and in subsequent sessions over various intervals, the EEG sensor unit 1035 can compare measurements with the baseline measurements. In some embodiments, the EEG sensor unit may include a transceiver or transmitter for sending information to controller 220 on the VNS stimulator 204 (FIG. 4). Also, in various embodiments, the EEG sensor unit 1035 may be coupled to a separate compact device (similar to compact device 812 in FIG. 8 as in the EKG embodiment), to mediate the flow of signals from the brain and to the EKG sensor unit 1035.

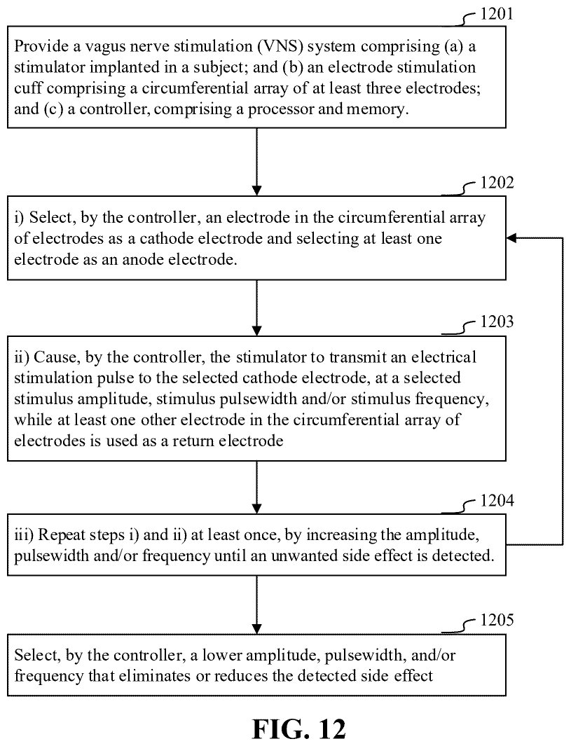

FIG. 12 is a conceptual flow diagram illustrating an exemplary method for providing VNS. As illustrated by this figure, such methods may comprise providing a VNS system comprising (a) a stimulator implanted in a subject; and (b) an electrode stimulation cuff comprising a circumferential array of at least three electrodes; and (c) a controller, comprising a processor and memory (1201), wherein the controller is performed to perform at least steps i), ii), and iii). In this example, the controller is configured to select, by the controller, an electrode in the circumferential array of electrodes as a cathode electrode and selecting at least one electrode as an anode electrode (1202), and cause the stimulator to transmit an electrical stimulation pulse to the selected cathode electrode, at a selected stimulus amplitude, stimulus pulse width and/or stimulus frequency, while at least one other electrode in the circumferential array of electrodes is used as a return electrode (1203). After transmitting this initial stimulus, the controller is configured to repeat steps 1202 and 1203 at least once, increasing the amplitude, pulse width and/or frequency until an unwanted side effect is detected (1204). In some cases, this repetition may occur once. However, in more typical cases multiple rounds of refinement will be needed. Side effects may be detected based upon feedback provided by the subject being treated or based on biomarker or physiological data obtained from the subject using one or more sensors (e.g., EMG or EKG sensors) communicatively linked to the VNS system. Upon detection of an unwanted side effect, the controller may then select a lower amplitude, pulse width and/or frequency for the stimulation that eliminates or reduces the unwanted side effect (1205). In another aspect, unipolar stimulation may be employed and the controller can be configured to select an electrode or electrodes as cathodes, while a conductive part of the stimulator housing is programmed or activated to operate as the return anode. The electrode in the cuff has at least two electrodes in this case.

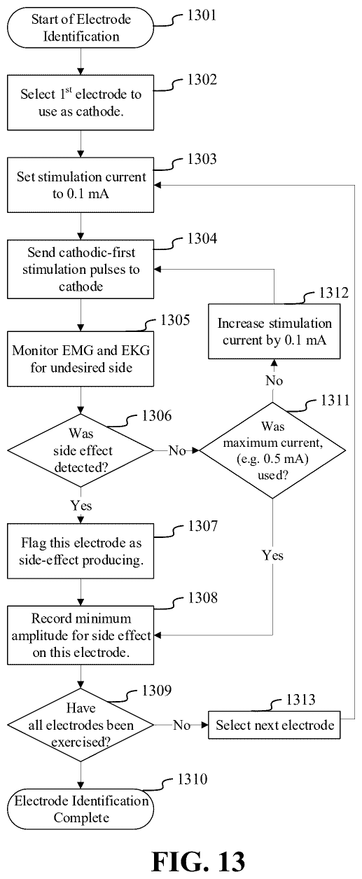

FIG. 13 is a conceptual flow diagram illustrating an exemplary method for selecting electrodes and stimulation parameters for the systems and methods described herein. As illustrated by this exemplary embodiment, the electrode identification process may start (1301) with the selection of an electrode to use as a cathode electrode (1302). For example, the controller of the VNS may select an electrode in a circumferential array of electrodes disposed on an inner surface, or within, the body of an electrode cuff used to stimulate a nerve (e.g., the vagus nerve) of a subject. In this example, the controller selected a stimulation current of 0.1 mA for the selected electrode (1303). Thereafter, cathodic first stimulation pulses are sent to the cathode at the selected stimulation current (1304). One or more sensors (e.g., an IMU, EEG, EMG, microphone-sound or EKG as shown here) may be used to monitor the subject to detect unwanted physiological side effects (1305). If an unwanted side effect is not detected (1306), the controller may then determine whether a maximum level for the selected stimulation parameter has been reached (e.g., 0.5 mA, as shown in this figure as the maximum stimulation current) (1311). If the maximum level has not been reached, the controller may then continue to use the selected electrode (1312), and return to administering stimulation (1304) once more. Alternatively, if an unwanted side effect is detected at step 1306, the controller may flag the selected electrode as a side-effect producing electrode (1307), and record the minimum level of the stimulation parameter that was found to induce the unwanted side effect on that electrode (1308). Amplitude was selected as the stimulation parameter in this example, however, other parameters such as pulse width and/or frequency may alternatively be used. As shown by this figure, step 1308 may also be reached when the controller determines that the maximum level for the selected stimulation parameter has been reached at step 1311. In either case, after this step, the controller may determine whether all electrodes have been tested (1309) and then either select the next electrode to test (1313) or, if no other electrodes remain to be tested, determine the electrode identification process has been completed (1310). The general procedure described for minimizing side-effects is applicable to both bipolar stimulation and unipolar stimulation.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones.

The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A system for vagus nerve stimulation (VNS), comprising:
a VNS stimulator implanted in a subject and configured to transmit electrical stimulation pulses to a vagus nerve of the subject using an electrode stimulation cuff comprising a circumferential array of electrodes; and
a controller comprising a processor and memory, configured to cause the VNS stimulator to transmit the electrical stimulation pulses to the vagus nerve by
a) selecting an electrode in the circumferential array of electrodes as a cathode electrode;
b) causing the stimulator to transmit an electrical stimulation pulse to the vagus nerve using the selected cathode electrode, while (i) at least one other electrode in the circumferential array of electrodes is used as a return electrode or (ii) a metallic housing of the VNS stimulator is used as an indifferent return anode; and
c) repeating steps a) and b) at least once, wherein a different electrode in the circumferential array of electrodes is selected as the cathode electrode.

2. The system of claim 1, wherein the circumferential array of electrodes comprises 2, 3, 4, 5, or 6 electrodes.

3. The system of claim 1, wherein the circumferential array of electrodes comprises 4 arc-shaped or semicircular electrodes evenly spaced along a circumference of the electrode stimulation cuff, when the electrode stimulation cuff is viewed cross-sectionally.

4. The system of claim 1, where each electrode in the circumferential array of electrodes is positioned along a circumference of the electrode stimulation cuff such that it is opposite to another electrode in the circumferential array of electrodes, when the electrode stimulation cuff is viewed cross-sectionally.

5. The system of claim 1, wherein the controller is further configured to use all other electrodes in the circumferential array of electrodes, other than the selected cathode electrode, as return electrodes when performing step b).

6. The system of claim 1, wherein the controller is configured to cause the stimulator to transmit the electrical stimulation pulses:
a) at different amplitudes, wherein each amplitude is independently determined for each selected cathode electrode;
b) pulses at different amplitudes until a physiologic response is detected; and/or
c) according to a periodic pattern.

7. The system of claim 1, wherein the controller is configured to cause the stimulator to increase the amplitude, pulse width and/or frequency of the electrical stimulation until an unwanted side effect is detected.

8. The system of claim 7, wherein the controller is further configured to record a minimum amplitude, pulse width and/or frequency of stimulation provided by a selected electrode that caused the unwanted side effect.

9. The system of claim 1, wherein the system further comprises on or more sensors configured to detect one or more physiological parameters of the subject being treated, and to determine whether the subject has experienced an unwanted side effect based on the detected one or more physiological parameters.

10. The system of claim 9, wherein the one or more sensors comprise an electrocardiogram (EKG) sensor, an electroencephalogram (ECG) sensor, and/or an electromyography (EMG) sensor.

11. A method for providing vagus nerve stimulation (VNS), comprising:
a) providing a VNS system comprising
a stimulator implanted in a subject,
an electrode stimulation cuff comprising a circumferential array of at least three electrodes, and
a controller, comprising a processor and memory, configured to cause the stimulator to transmit the electrical stimulation pulses to a nerve of the subject by
a) selecting an electrode in the circumferential array of electrodes as a cathode electrode and one of (i) selecting at least one electrode as an anode electrode, or (ii) selecting a metal housing part of the stimulator as the return anode;
b) causing the stimulator to transmit an electrical stimulation pulse to the selected cathode electrode, at a selected stimulus amplitude, stimulus pulsewidth and/or stimulus frequency, while at least one other electrode in the circumferential array of electrodes is used as a return electrode or the metal housing part of the stimulator is used as the return electrode; and
c) repeating steps a) and b) at least once, by increasing the amplitude, pulsewidth and/or frequency until an unwanted side effect is detected, and then selecting a lower amplitude, pulsewidth, and/or frequency that eliminates or reduces the detected side effect, wherein the stimulus amplitude may be based on either constant current or constant voltage stimulus.

12. The method of claim 11, wherein the selection of the cathode electrode is based on data obtained from monitoring the subject following prior electrical stimulation using the VNS system.

13. The method of claim 12, wherein the data comprises information describing one or more undesired side effects experienced by the subject following the prior electrical stimulation.

14. The method of claim 13, wherein the one or more undesired side effects comprise laryngeal EMG activation, tachycardia, or bradycardia.

15. The method of claim 11, wherein the circumferential array of electrodes comprises 2, 3, 4, 5, or 6 electrodes.

16. The method of claim 11, wherein the circumferential array of electrodes comprises 4 arc-shaped or semicircular electrodes evenly spaced along a circumference of the electrode stimulation cuff.

17. The method of claim 11, where each electrode in the circumferential array of electrodes is positioned along a circumference of the electrode stimulation cuff such that it is opposite to another electrode in the circumferential array of electrodes.

18. The method of claim 11, wherein the controller is configured to receive user input from the subject regarding the occurrence of unwanted side effects.

19. The method of claim 11, wherein the controller is configured to detect whether an unwanted side effect has occurred based on one or more physiological parameters of the subject determined using at least one sensor communicatively-linked with the controller.

20. The method of claim 19, wherein the at least one sensor comprises an EMG sensor, an EKG sensor, or an ECG sensor.

21. The method of claim 11, wherein the circumferential array of electrodes comprises 4 arc-shaped or semicircular electrodes evenly spaced along a circumference of the electrode stimulation cuff, when viewed cross-sectionally; and
wherein the stimulator is configured to automatically deliver to the chosen cathode electrode incremental increases of the stimulus amplitude or pulsewidth to thereby select a lower amplitude or pulsewidth that eliminates or substantially reduces unwanted side effects.
